(19) United States
(12) Patent Application Publication
(10) Pub. No.: US 2014/0212957 A1
(43) Pub. Date: Jul. 31, 2014

(54) PRODUCTION OF OPTICALLY PURE PROPANE-1,2-DIOL

(71) Applicants: Wolfgang Tietz, Biendorf (DE); Joachim Schulze, Soest (DE); Armin Boerner, Rostock (DE); Ivan Shuklov, Rostock (DE); Klaus Kuehlein, Kelkheim (DE)

(72) Inventors: Wolfgang Tietz, Biendorf (DE); Joachim Schulze, Soest (DE); Armin Boerner, Rostock (DE); Ivan Shuklov, Rostock (DE); Klaus Kuehlein, Kelkheim (DE)

(73) Assignee: THYSSENKRUPP INDUSTRIAL SOLUTIONS GMBH, Essen (DE)

(21) Appl. No.: 14/233,839

(22) PCT Filed: Jun. 22, 2012
(86) PCT No.: PCT/EP2012/002638

§ 371 Date: Apr. 8, 2014

(30) Foreign Application Priority Data
Jul. 20, 2011 (DE) 10 2011 107 959.2

(51) Int. Cl. C07C 29/74; C07C 31/20; C12P 41/00; C07C 29/149

(52) U.S. Cl. CPC C07C 29/74; C07C 29/149; C07C 31/205; C12P 41/004
USPC 435/280

(57) ABSTRACT

A method for producing optically pure propane-1,2-diol, including the method steps: a. hydrogenation of lactides, metal-catalysed heterogenous catalysis being carried out in the presence of hydrogen, a crude product containing propane-1,2-diol being produced, and b. dynamic, kinetic racemate resolution, propane-1,2-diol of an optical purity in the range of ≧99% e.e. being produced.

The invention relates to a process for the production of optically pure propane-1,2-diol from lactides.

Propane-1,2-diol is produced on an industrial scale by means of the hydrolysis of propylene oxide, or from glycerine. It is predominantly used in cosmetic products, such as skin creams and toothpaste. It improves the absorption of different active ingredients and demonstrates antimicrobial efficacy. Furthermore it is an approved food additive in the EU. It is also used as a carrier and a carrier solvent for colourants, antioxidants and emulsifiers.

Lactides in this instance are cyclical diesters of lactic acid. During lactic acid polymerisation, for example, different types of lactides can occur. These can be pure L,L-lactide or pure D,D-lactide. As a result of the prevailing high temperatures required for a rapid reaction process, and due to the cationic contaminants in the lactic acid or the reaction vessels (e.g. caused by corrosion), the problem of racemisation arises whereby meso-lactide is formed as a by-product. Like L,L-lactide, meso-lactide is a cyclical diester with two optically active carbon atoms in the ring. It has an optical R and an S centre and is consequently optically inactive. Meso-lactides have a negative impact on an associated lactic acid polymerisation and have to be separated off. Consequently they are produced as a by-product of lactic acid polymerisation.

Furthermore, there are rac-lactides and these are yielded from the same amounts of D,D-lactide and L,L-lactide by means of melting, for example. The individual lactides can be differentiated by their melting temperatures. The L,L-lactide and the D-D-lactide have a melting temperature of 97° C., whilst the meso-lactide has a melting temperature of 54° C., and the L,L/D,D-lactide has a melting temperature of 129° C.

The hydrogenation of the alkyl esters from lactic acid to form propane-1,2-diol is known. This transformation is possible with both heterogeneous catalysts and homogeneous catalysts.

The hydrogenation of lactic acid ethyl ester was described in ethanol using a copper-oxide-chrome-oxide catalyst at 125° C. and H₂ pressure of 345 bar, for example (H. Adkins et al, J. Am. Chem. Soc. 1948, 70, 3121-3125). The use of a copper oxide-chrome-oxide-barium catalyst at 250° C. and 300 bar hydrogen pressure was also successful (K. Folkers et al, J. Am. Chem. Soc. 1932, 54, 1145-1154). Just recently the hydrogenation of lactic acid esters using copper silicates in the gas phase was described in WO 2011036189 A1 and WO 2009103682 A1. Copper on aluminium oxide was also suggested in WO 2005023737 A1 for the reduction of lactic acid methyl esters.

Furthermore a range of heterogeneous ruthenium catalysts has also been investigated. For example, Ru—B supported on titanium oxide is an active catalyst for the hydrogenation of lactic acid ethyl esters in water as the solvent at 90° C. and 40 bar H₂ (G.-Y. Fan et al, Chem. Lett. 2008, 37, 852-853). The catalyst was prepared by reducing RuCl₃ using NaBH₄. RuB on a tin-modified SBA-15 molecular sieve (G. Luo et al, Appl. Catal., A: General 2007, 332, 79-88) and Ru—B on y-aluminium oxide (G. Luo et al, J. Mol. Catal. A: Chemical 2005, 230, 69-77 and G. Luo et al, Appl. Catal., A: General 2004, 275, 95-102) also led to average to good yields in the reduction of lactic acid ethyl esters. Unfortunately, the Ru—B catalysts are not chemoselective. A Nishimura catalyst (Rh/Pt-oxide) proved itself to be efficient in the hydrogenation of lactic acid ethyl esters at 25° C. and 100 bar hydrogen pressure in MeOH (M. Studer et al, Adv. Synth. Catal. 2001, 343, 802-808). Homogeneous ruthenium catalysts with modifying P,N-ligands (EP2161251 A1; W. Kuriyama et al, Adv. Synth. Catal. 2010, 352, 92-96) or P,P-ligands (EP 1970360 A1) were used very successfully in the hydrogenation of lactic acid esters, wherein the reactions occurred at temperatures of 80-90° C. and H₂ pressures of 30-50 bar H₂.

Only recently did the reduction of lactides to propanediol-d₂ succeed using lithium aluminium deuteride within the framework of mechanistic studies (R. M. Painter et al, Angew. Chem. Int. Ed. 2010, 49, 9456-9459).

WO2006/124899 describes the catalytic hydrogenation of lactides to propylene glycol. In this instance the hydrogenation is carried out either in the gas phase or in the liquid phase in the presence of aliphatic alcohols, for example. In so doing reaction conditions of 20° C. to 250° C. and 1.4 to 275 bar are taken as a basis, and the reaction time is 1 to 10 hours. With this reaction it makes no difference whether the starting product is one of the enantiomers or a mixture of them. It can, however, be assumed that racemisation occurs during the reaction and that the propylene glycol is therefore not obtained in an optically pure form.

This is disadvantageous for many applications as although both enantiomers have the same physical properties, they both react differently in chemical reactions in which another enantiopure reaction partner is involved. Equally when used in the field of pharmacology and in applications in the fields of agricultural chemistry, odours and flavours, enantiomeric substances cause different effects with each other.

To obtain an enantiomer in its optically pure form from racemic mixtures dynamic kinetic racemic resolution (DKR) is known. Only very small amounts of an Ru catalyst (up to 0.05 mol %) are required to achieve the racemic resolution of alcohols (K. Bogar et al, Beilstein J. Org. Chem 2007, 3 (50)), this being a kinetic racemic resolution with in situ racemisation of the substrate. The racemic resolution occurs enyzmatically by means of biocatalysis, and racemisation is achieved by means of metal catalysts, but also by means of organo-catalysts, bases, heating, the use of enzymes, Lewis acids, and redox and radical reactions. The application of the process for the production of propane-1,2-diol in an optically pure form from lactides is, however, not known.

For this reason it would be preferable to provide a process which permits propane-1,2-diol to be generated in an optically pure form. Furthermore, this process should originate from lactides, particularly as meso-lactide is obtained as a waste product in lactic acid polymerisation and could, therefore, be put to other uses. However, the other lactide forms mentioned above could also be converted advantageously to optically pure propane-1,2-diol.

Therefore, the objective of the invention is to provide a process which enables optically pure propane-1,2-diol to be produced from lactides within a range of ≧99% e.e.

The invention achieves this objective by means of a process for the production of optically pure propane-1,2-diol comprising the following process steps:
a. Hydrogenation of lactides wherein a metal-catalysed heterogeneous catalysis is carried out in the presence of hydrogen, a raw product containing propane-1,2-diol being produced, and
b. Dynamic kinetic racemic resolution, in which optically pure propane-1,2-diol is produced within a range of ≧99% e.e..

In the process the following reaction occurs in step a):

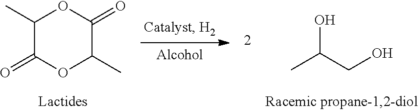

The alcohol functions as both a solvent and a reactant, the concentration of lactide in the alcohol being uncritical in terms of the yield obtained. The alcohol should preferably be available in excess.

The system used for dynamic kinetic racemic resolution comprises a catalyst which adjusts the upstream racemisation balance, and an enzyme that extracts one of the enantiomers from the racemisation balance by means of esterification.

The term “optically pure” within the context of this application means enantiopure propane-1,2-diol. That means that the production of >99% e.e. optically pure propane-1,2-diol, as provided for in the principal claim, can be equated to 99% enantiopurity. Whether the (R)-enantiomer or the (S)-Enantiomer is produced is of no significance.

In one embodiment of the process according to the invention lactides selected from the group comprising D,D-lactide, L, L-lactide, meso-lactide and L,L/D,D-lactide are used. The lactides are cyclical esters of lactic acids which can occur in the form of enantiomers, i.e. in D or L form. L,L-lactide describes an ester comprising two L-lactic acids and is also referred to as S,S-lactide in specialist literature. The same applies to the D,D-lactide, which is also referred to as R,R-lactide. L,L/D,D-lactide is understood to mean the racemate (also referred to in specialist literature as rac-lactide or R,S-lactide) comprising the equimolar mixture of D,D-lactide and L,L-lactide. In contrast, meso-lactide describes a lactide comprising D- and L-lactic acid. Claim 2, therefore, demonstrates that all possible lactides can be subjected to the process according to the invention. This also includes oligolactides with different lactic acid enantiomer compositions, and preferably dilactides.

It is advantageous to carry out the metal-catalysed heterogeneous catalysis in the liquid phase in step a). In so doing preference is given to selecting the liquid phase from a group of solvents comprising water, aliphatic or aromatic hydrocarbons with a chain length of up to 10 C-atoms, and mixtures thereof, wherein the aliphatic hydrocarbons are preferably alcohols with particular preference being given to methanol and/or ethanol being used.

In a preferred embodiment of the process according to the invention the heterogeneous catalysis in step a) is carried out by means of a catalyst from the metals group, wherein the metal is selected from a group comprising ruthenium, rhodium, rhenium, palladium, platinum, nickel, cobalt, molybdenum, wolfram, titanium, zirconium, niobium, vanadium, chromium, manganese, osmium, iridium, iron, copper, zinc, silver, gold, barium and mixtures thereof, preference being given to copper-chromite catalysts and/or copper-chromite catalysts with added barium.

In additional embodiments of the process the heterogeneous catalysis in step a) is carried out at a hydrogen pressure of less than 20 to 300 bar, with preference given to a hydrogen pressure of less than 130 to 170 bar, and particular preference given to a hydrogen pressure of less than 140 to 160 bar.

The heterogeneous catalysis in step a) is preferably carried out within a temperature range of 20° C. to 250° C., preferably within a temperature range of 130° C. to 170° C., with particular preference given to a temperature range of 145° C. to 155° C.

As an option, prior to the heterogeneous catalysis being carried out in step a), the pressure vessel is rinsed 1 to 5 times, preferably 3 times, with hydrogen.

In a further embodiment of the process the heterogeneous catalysis is carried out in step a) over a period of 5 to 20 hours, preferably over a period of 10 to 18 hours, with particular preference given to a period of 12 to 16 hours.

It is advantageous to agitate during the heterogeneous catalysis in step a). It is also advantageous for hydrogen to be continuously pushed through during the heterogeneous catalysis in step a).

In preferred embodiments of this process the catalyst is separated off from the raw product once the heterogeneous catalysis in step a) has been completed.

In a further embodiment the raw product resulting from step a) is subjected to a concentration step and/or a distillation step, wherein a fraction containing propane-1,2-diol and a fraction containing solvent are generated.

It is preferred that the solvent, which is used in the heterogeneous catalysis in step a), is fed back into the process.

In a further design variant of the process, the propane-1,2-diol, which is obtained from step a), is furnished with a protective group and 1-O-substituted propanediol is produced. It is advantageous for the protective group to be a recyclable, achiral protective group and is selected from the group comprising tert-butyl, phenyl, methyl, acetyl, benzoyl, trityl, silyl and benzyl. This means that pivalates, p-methoxybenzyl, trimethylsilyl, triethylsilyl, triisopropylsilyl, diphenylmethylsilyl or di-tert-butylmethylsilyl can be used. In principle any achiral protective group can be used (T. W. Green et al, Protective Groups in Organic Synthesis, Wiley-Interscience, New York, 1999). Particular preference is given to the protective group tert-butyl of the primary hydroxyl group of the propane-1,2-diol from step a).

In a further embodiment an enzymatic racemic resolution is used for the dynamic kinetic racemic resolution in the presence of a metal catalyst during step b). Preference is given to using lipases. Ruthenium catalysts are the preferred metal catalysts. Particular preference is given to ruthenium catalysts with immobilised lipases.

The dynamic kinetic racemic resolution in step b) is preferably carried out within a temperature range of 60° C. to 90° C. In so doing, the reaction time is 30 to 200 hrs, preferably 40 to 60 hrs.

In a further embodiment the dynamic kinetic racemic resolution in step b) is carried out in the presence of Na₂CO₃, the Na₂CO₃ being added in a quantity of 0.4 mmol to 5 mmol per 33 mg enzyme, which corresponds to 330 units. Na₂CO₃ is practically insoluble in the reaction medium and acts as a heterogeneous additive. The most advantageous enzyme for this is Novozym 435.

The present invention is explained in more detail below using several embodiment examples.

### EXAMPLE 1

### Hydrogenation of the Rac-Lactide using a Cu/Cr Catalyst

L,L/D,D-lactide (1.00 g, 6.9 mmol) and copper chromite (1.33 g, 133 wt %) are suspended in 5 ml abs. MeOH in a 10 ml autoclave. The autoclave is rinsed three times with H₂. 150 bar hydrogen pressure is then applied. The reaction mixture is stirred for 15 hours at 150° C. The hydrogen is continuously pressed through, a pressure of between 148 and 153 bar being maintained. After the autoclave has been cooled and aired the reaction mixture is diluted using 5 ml MeOH and centrifuged off from the catalyst (75 min, 4,500 rpm). The blue-green reaction solution is decanted, the residue is washed with 3 ml MeOH, and concentrated in a vacuum at 40° C. and 40 mbar. The raw product (2.06 g) has a dark blue colour and comprises propane-1,2-diol contaminated with approximately 5% MeOH (¹³C-NMR spectrum). The pure product (0.68 g, 68%) is obtained as a colourless liquid after distillation at 101-102° C. and 8 mbar. After distillation the inorganic residue amounts to approximately 30 mg.

### EXAMPLE 2

### Hydrogenation of the Rac-Lactide using a Cu/Cr/Ba Catalyst

L,L/D,D-lactide (1.00 g, 6.9 mmol) and copper chromite (1.33 g, 133 wt %) doped with barium are suspended in 5 ml abs. MeOH or EtOH in a 10 ml autoclave. The autoclave is rinsed three times with H₂. 150 bar hydrogen pressure is then applied. The reaction mixture is stirred for 12 hours at 150° C. The hydrogen is continuously pushed through, a pressure of between 148 and 153 bar being maintained. After the autoclave has been cooled and aired the reaction mixture is diluted with 5 ml MeOH and the catalyst is centrifuged off (15 min, 4,500 rpm). The reaction solution is concentrated in a vacuum at 40° C. and 40 mbar. The raw product is light blue in colour and comprises propane-1,2-diol which is still contaminated with approximately 5% MeOH. This was determined via a ¹³C-NMR spectrum (not shown). The pure product (0.8 g, 82%) is obtained as a colourless liquid by means of distillation at 101-102° C. and 8 mbar. The reaction with EtOH takes place at a considerably slower pace than in MeOH.

The advantage of the Cu/Cr/Ba catalyst is that the reaction takes place more quickly compared to the Cu/Cr catalyst. This was determined via hydrogen consumption curves which were recorded during tests. From this it followed that hydrogenation takes place approximately 20% more quickly with the Cu/Cr/Ba catalyst. Furthermore, practically none of the catalyst dissolves in the reaction solution when a Cu/Cr/Ba catalyst is used which means that the reaction is completely heterogeneous. In contrast, up to 30 mg out of a total quantity of 1.3 g Cu/Cr catalyst were contained in the reaction solution following a hydrogenation trial.

### EXAMPLE 3

### Hydrogenation of Additional Lactide Forms using a Cu/Cr/Ba Catalyst

The method corresponded to that described in Example 2 in the presence of 5 ml MeOH at 150 bar H₂ and using the Cu/Cr/Ba catalyst. The exact reaction conditions are shown in Table 1.

TABLE 1

| | | Starting | | | |
|---|---|---|---|---|---|
| | | Quantity | Time | Temperature | GC Yield |
| Run | Substrate | [g] | [h] | [° C.] | [%] |
| 1 | rac-lactide | 1.0 | 15 | 150 | 100 |
| 2 | L,L-lactide | 1.0 | 15 | 150 | 100 |
| 3 | meso-lactide | 1.0 | 15 | 150 | 100 |

Table 1 shows that all forms of lactide, including meso-lactide, which are obtained as waste product during lactic acid polymerisation, can be 100% converted. This means that the process according to the invention is suitable for converting meso-lactides to propane-1,2-diol. Meso-lactide, that was still contaminated with residues of lactic acid, was not able to be converted to propane-1,2-diol. For this reason it is necessary to use the lactides in their pure or purified form for hydrogenation.

### EXAMPLE 4

### Examination of the Racemisation Degree of the Propane 1,2-diol Produced by means of Hydrogenation

To derivatise the propane-1,2-diol produced by the hydrogenation processes 0.28 g (3.7 mmol) propane-1,2-diol were added to 1.2 ml phenylisocyanate (11 mmol). The reaction mixture was heated for 30 mins at 100° C. and then cooled to room temperature. Diethyl ether (5 ml) was then added. The white crystals produced were filtered off and washed with 50 ml hexane. The resulting product was used for analysing the entantiomers, to which end it was separated in a CHIRALCEL®OD-H chiral HPLC column into heptane/EtOH 80:20.

The results obtained when using L,L-lactide, which was produced according to the instructions in Example 2, are shown in Table 2.

TABLE 2

| | | Starting | | | | |
|---|---|---|---|---|---|---|
| | | Quantity | Time | Temperature | Yield | e.e. |
| Run | Substrate | [g] | [h] | [° C.] | [%] | [%] |
| 1 | L,L-lactide | 1.0 | 12 | 125 | 90 | 88 |
| 2 | L,L-lactide | 0.5 | 12 | 150 | 100 | 0 |

Table 2 shows that the enantiomeric purity of the propanediol resulting from the hydrogenation process is dependent upon the temperature. At a temperature of 150° C. only a racemic mixture is obtained. At 125° C. the e.e. value is 88%. Therefore, a racemic mixture of propane-1,2-diol occurs during the hydrogenation of the lactides. If the temperature is lowered any further there is a risk that the hydrogenation reaction will come to a standstill.

### EXAMPLE 5

### Dynamic Kinetic Racemic Resolution for the Production of Optically Pure Propane-1,2-diol

By way of example, tert-butyl was introduced as the protective group and tert-butyloxypropane-2-ol was obtained from the racemic mixture of propane-1,2-diol which was obtained through the hydrogenation process. The enzymatic racemic resolution occurs according to the following diagram:

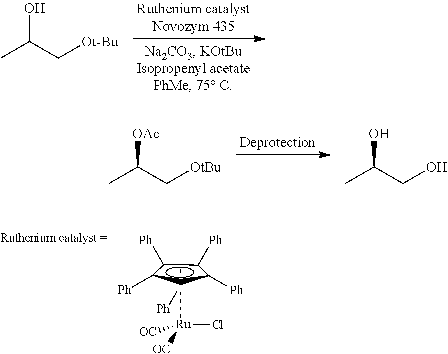

The reaction was carried out in 7.5 ml toluene at 75° C. 20 mmol isopropenyl acetate, 19.8 mmol 1-tert-butoxypropanol-2, 0.02 mmol (Ph₅Cp)Ru(CO)₂Cl, 0.04 mmol t-BuOK, 50 mg Na₂CO₃were admixed. The results are shown in Table 3:

TABLE 3

| | Time | Novozym 435 | Yield | e.e. |
|---|---|---|---|---|
| Run | [h] | [mg] | [%] | [%] |
| 1 | 68 | 13 | 60 | 99 |
| 2 | 42 | 33 | 66 | 99 |
| 3 | 90 | 33 | 80 | 99 |
| 4 | 190 | 33 | 85 | 99 |

Table 3 show that as little as 13 mg Novozym 435 (Run 1) is sufficient to produce excellent stereoselectivity of >99% e.e.. However, the yield was to be increased further, so 2.5 times the amount of enzymes was used. (Runs 2-4). It was observed that although the ruthenium-catalysed epimerisation slows down with larger quantities of enzyme, the yield increases.

### EXAMPLE 6

### Dynamic Kinetic Racemic Resolution for Producing Optically Pure Propane-1,2-diol with Further Improved Yield

The reaction was carried out in 20 ml toluene at 75° C. 20 mmol isopropenyl acetate, 19.8 mmol 1-tert-butoxypropano1-2, 0.06 mmol (Ph₅Cp)Ru(CO)₂Cl, Novozym 435 33 mg, 0.1 mmol t-BuOK were mixed in. To investigate the influence of Na₂CO₃ on the reaction's yield, the concentration of Na₂CO₃ was varied. The results are shown in Table 4:

TABLE 4

| | Time | Na₂CO₃ | Yield | e.e. |
|---|---|---|---|---|
| Run | [h] | [mg] | [%] | [%] |
| 1 | 48 | 50 | 65 | 99 |
| 2 | 120 | 50 | 85 | 99 |
| 3 | 48 | 150 | 85 | 99 |
| 4 | 120 | 150 | 92 | 99 |

Table 4 shows that the reaction is considerably quicker in the presence of larger quantities of the base Na₂CO₃. Consequently a yield of 65% can be achieved after 48 hours in the presence of 50 mg (Run 1), whilst with 150 mg Na₂CO₃ and the same amounts of catalyst and enzyme a yield of 85% can be achieved (Run 4).

### EXAMPLE 7

### Dynamic Kinetic Racemic Resolution of 1-tert-butoxypropanol-2 Measured in Grams

Chlorodicarbonyl(1,2,3,4,5-pentaphenylcyclopentadienyl)ruthenium (40 mg, 0.06 mmol), immobilised CALB from Aldrich (33 mg), and Na₂CO₃ (0.15 g, 1.4 mmol) were added to a 50 ml Schlenk vessel with a magnetic agitator. The vessel was evacuated and filled with argon. Toluene (20 ml) was added to an argon atmosphere. The reaction mixture was stirred at room temperature until the ruthenium complex dissolved. A solution of ^{t}BuOK in THF (1 M) (0.1 ml, 0.1 mmol) was then added and the reaction mixture was stirred for a further 6 minutes. 1-tert-butoxypropanol-2 (2.62 g, 3 ml, 19.8 mol) was added to the resulting mixture and the reaction mixture was stirred for a further 4 minutes. Isopropenyl acetate (2.00 g, 20 mol) was then added at room temperature and the reaction mixture was heated to 75° C. A sample was taken after 120 hrs and analysed with the help of the GC (HP-5, 50 m). According to this analysis a yield of 93% was achieved. The reaction mixture was then cooled, filtered through a paper filter, and concentrated at a reduced pressure of 20 mbar. The residue was distilled in a vacuum (80° C., 5 mbar). (R)-2-O-acetyl-1-O-tert-butyl-propane-1,2-diol 2.15 g (63% yield, 99.5% e.e.) was obtained as a colourless liquid.

Advantages associated with the process according to the invention:
Production from lactides (production from meso-lactides is also possible) of propane-1,2-diol with an optical purity of >99% e.e. which is produced as a waste product during lactic acid polymerisation

1. Process for the production of optically pure propane-1,2-diol comprising the following process steps:
Hydrogenation of lactides, wherein a metal-catalysed heterogeneous catalysis is carried out in the presence of hydrogen, a raw product containing propane-1,2-diol being produced, and
Dynamic kinetic racemic resolution, in which optically pure propane-1.2-diol is produced within a range of ≧99% e.e.

2. Process in accordance with claim 1, wherein the lactides are selected from the group comprising D,D-lactide, L,L-lactide, meso-lactide and L,L/D,D-lactide.

3. The process in accordance with claim 1, wherein the metal-catalysed heterogeneous catalysis in step a) is carried out in the liquid phase.

4. Process in accordance with claim 3, wherein the liquid phase is selected from a group of solvents comprising water, aliphatic or aromatic hydrocarbons with a chain length of up to 10 C-atoms, and mixtures thereof, wherein the aliphatic hydrocarbons are preferably alcohols with particular preference being given to methanol and/or ethanol being used.

5. The process in accordance with claim 1 wherein the heterogeneous catalysis in step a) is carried out using a catalyst from the metals group, wherein the metal is selected from a group comprising ruthenium, rhodium, rhenium, palladium, platinum, nickel, cobalt, molybdenum, wolfram, titanium, zirconium, niobium, vanadium, chromium, manganese, osmium, iridium, iron, copper, zinc, silver, gold, barium and mixtures thereof, preference being given to the use of copper-chromite catalysts and/or copper-chromite catalysts with barium added.

6. The process in accordance with claim 1, wherein the heterogeneous catalysis in step a) is carried out at a hydrogen pressure of less than 20 to 300 bar, with preference given to a hydrogen pressure of less than 130 to 170 bar, and particular preference given to a hydrogen pressure of 140 to 160 bar.

7. The process in accordance with claim 1, wherein the heterogeneous catalysis in step a) is carried out within a temperature range of 20° C. to 250° C., preferably within a temperature range of 130° C. to 170° C., with particular preference given to a temperature range of 145° C. to 155° C.

8. The process in accordance with claim 1, wherein prior to the heterogeneous catalysis in step a) being carried out, the pressure vessel is rinsed 1 to 5 times, preferably 3 times, with hydrogen.

9. The process in accordance with claim 1, wherein the heterogeneous catalysis is carried out in step a) over a period of 5 to 20 hours, preferably over a period of 10 to 18 hours, with particular preference given to a period of 12 to 16 hours.

10. The process in accordance with claim 1, wherein agitation occurs during the heterogeneous catalysis in step a).

11. The process in accordance with claim 1, wherein hydrogen is continuously pushed through during the heterogeneous catalysis in step a).

12. The process in accordance with claim 1, wherein the catalyst is separated off from the raw product once the heterogeneous catalysis in step a) has been completed.

13. The process in accordance with claim 1, wherein the raw product resulting from step a) is subjected to a concentration step and/or a distillation step, wherein a fraction containing propane-1,2-diol and a fraction containing solvent are generated.

14. The process in accordance with claim 1, wherein the solvent used for the heterogeneous catalysis in step a) is fed back into the process.

15. The process in accordance with claim 1, wherein the propane-1,2-diol, which is obtained from step a), is furnished with a protective group and 1-O-substituted propanediol is produced.

16. Process in accordance with claim 15, wherein the protective group is a recyclable, achiral protective group and is selected from the group comprising tert-butyl, phenyl, methyl, acetyl, benzoyl, trityl, silyl and benzyl.

17. The process in accordance with claim 1, wherein an enzymatic racemic resolution is used for the dynamic kinetic racemic resolution in the presence of a metal catalyst during step b).

18. The process in accordance with claim 17, wherein lipases are used during the enzymatic racemic resolution.

19. The process in accordance with claim 17, wherein ruthenium catalysts are used as metal catalysts.

20. The process in accordance with claim 17, wherein the dynamic kinetic racemic resolution is carried out using ruthenium catalysts with immobilised lipases.

21. The process in accordance with claim 1, wherein the dynamic kinetic racemic resolution is carried out in step b) within a temperature range of 60° C. to 90° C.

22. The process in accordance with claim 1, wherein the dynamic kinetic racemic resolution in step b) is carried out over a period of 30 to 200 hrs, preferably within a period of 40 to 60 hrs.

23. The process in accordance with claim 1, wherein the dynamic kinetic racemic resolution in step b) is carried out in the presence of Na₂CO₃, whereby the Na₂CO₃ is added in quantities of 0.4 mmol to 5 mmol per 33 mg of enzyme.
